(19) United States
(12) Patent Application Publication
(10) Pub. No.: US 2022/0072158 A1
(43) Pub. Date: Mar. 10, 2022

(54) ISL1-BASED GENE THERAPY TO TREAT HEARING LOSS

(71) Applicant: Massachusetts Eye and Ear Infirmary, Boston, MA (US)

(72) Inventors: Zheng-Yi Chen, Somerville, MA (US); Yujuan Hu, Boston, MA (US)

(21) Appl. No.: 17/455,135

(22) Filed: Nov. 16, 2021

Related U.S. Application Data
(63) Continuation of application No. 16/096,980, filed on Oct. 26, 2018.
(60) Provisional application No. 62/327,639, filed on Apr. 26, 2016.

(51) Int. Cl. A61K 48/00; A61K 38/17; C12Q 1/6883; A61K 45/06; A61K 9/00; A61P 27/16

(52) U.S. Cl. CPC A61K 48/0058; A61K 38/1709; C12Q 1/6883; A61K 45/06; C12N 2750/14143; A61P 27/16; A61K 48/0075; C12Q 2600/158; G01N 2800/14; A61K 9/0046

(57) ABSTRACT

Compositions for the prevention, treatment and/or reversal of hearing loss include vectors encoding an Islet-1 (Isl1) nucleic acid sequence. The over-expression of Isl1 molecules in ear cells, for example, hair cells, results in the treatment of hearing loss due to age, noise exposure or any idiopathic causes.

### FIELD OF THE INVENTION

Embodiments of the invention are directed to compositions for delivery of Islet-1 (Isl1) molecules into mammalian outer and/or inner ear hair cells. Methods of use include administering to a subject these Isl1 compositions to prevent, treat and reverse hearing loss due to aging, noise, idiopathic factors, or any other factors.

### BACKGROUND

Hearing loss affects hundreds of millions of people worldwide. The most common form of hearing loss is age-related hearing loss (ARHL) or presbycusis. ARHL affects over 50% of the population older than 75 years of age. In addition to the difficulties in communication, ARHL has been associated with declines in other aspects of health including dementia, depression and balance. The causes of ARHL are likely to be multi-factors including environmental exposure, genetic predisposition and aging. The precise contribution of each factor to ARHL is unknown. The medical intervention is confined to hearing aids, which is of limited value especially in patients with deficits in speech recognition. Studies of human temporal bones have classified ARHL to distinct categories including defects in hair cells, neurons, stria and mechanical. While it is generally accepted that hair cell loss may be a leading cause of ARHL, increasing evidence has also pointed to the important roles of diminished connections between hair cells and neurons, the synapses, in the onset and progression of ARHL (Kujawa, S. G. & Liberman, M. C. J. Neurosci. 26, 2115-2123 (2006)). Given the prevalence of ARHL and the increase in aging populations, there is an urgent need to develop new treatment for ARHL.

Studies have shown that genetic predispositions play important roles in ARHL. Dominant non-syndromic deafness is generally manifested as progressive hearing loss, with variable ages of onset and the time frames of disease progression. A mutation in a ATP-gated receptor P2X2 has been found to be responsible for ARHL, which is exacerbated after noise exposure (Yan, D. et al. Proc. Natl. Acad. Sci. U.S.A. 110, 2228-2233 (2013)). However, GWAS (genome-wide association study) have so far failed to identify genes responsible for a majority of human ARHL (Friedman, R. A. et al. Hum. Mol. Genet. 18, 785-796 (2009)), an indication of the complexity and heterogeneities involved. In animal models, a number of genes have been identified that are associated with ARHL including Cdh23 and Fscn2 with important roles in hair cell stereocilia function and mt-Tr in mitochondria function (Keithley, E. M., et al. Hear. Res. 188, 21-28 (2004); Shin, J.-B., et al. J. Neurosci. 30, 9683-9694 (2010); Johnson, K. R., et al. Nature genetics 27, 191-194 (2001)).

### SUMMARY

Embodiments of the invention are directed to delivery of Islet-1 (Isl1) molecules into mammalian outer and/or inner ear cells to prevent, treat and reverse hearing loss due to aging, noise exposure, idiopathic causes and any other physiological or physical factors.

In certain embodiments, a viral delivery system is constructed to deliver an Isl1 gene, mutants, variants or fragments thereof into mammalian outer and/or inner ear cells to prevent, treat and/or reverse hearing loss due to aging, noise exposure, idiopathic and other factors.

In some embodiments, a composition comprises a vector encoding an Islet-1 (Isl1) nucleic acid sequence wherein the Isl1 nucleic acid sequence is under control of a tissue specific promoter sequence. The tissue specific promoter can be a constitutive or inducible promoter e.g. CMV. In one embodiment, the tissue specific promoter sequence is a hair-cell specific promoter sequence.

In other embodiments, a vector comprises: a lentivirus vector, an adenovirus vector, an adeno-associated virus (AAV) vector, a vesicular stomatitis virus (VSV) vector, a herpes simplex virus (HSV) vector, a vaccinia virus vector, a pox virus vector, an influenza virus vector, a respiratory syncytial virus vector, a parainfluenza virus vector, a foamy virus vector, a retrovirus vector, a eukaryotic vector or a plasmid.

In some embodiments, the vector is a viral vector comprising capsid polypeptides having a lower seroprevalence in a subject as compared to the wild-type virus.

In other embodiments, a method of preventing, treating and/or reversing age-related hearing loss, noise-induced hearing loss or idiopathic hearing loss in a subject in need thereof, the method comprises administering to an outer and/or inner ear cell of the subject, a virus vector comprising an Islet-1 (Isl1) nucleic acid sequence wherein the Isl1 is overexpressed in the outer and/or inner ear cells as compared to expression of Isl1 in a normal outer or inner ear cell; and/or, administering cells comprising a vector encoding an Islet-1 (Isl1) nucleic acid sequence; and/or an Isl1 molecule; and/or agents comprising small molecules that activate Isl1 in inner ear cells including hair cells. In certain embodiments, inner car cells comprise: stria vascularis, hair cells, supporting cells or ganglion neurons.

In some embodiments, the Isl1 nucleic acid sequence is under control of a tissue specific promoter sequence wherein the promoter is a constitutive or inducible promoter. In some embodiments, the tissue specific promoter sequence is a hair-cell specific promoter sequence, a stria vascularis specific promoter sequence or a supporting cell specific promoter sequence or a ganglion neuron specific promoter sequence.

In certain embodiments, the virus vector comprises: a lentivirus, an adenovirus, an adeno-associated virus (AAV), a vesicular stomatitis virus (VSV), herpes simplex virus (HSV), vaccinia virus, pox virus, influenza virus, respiratory syncytial virus, parainfluenza virus, foamy virus or a retrovirus. In embodiments, a vector or delivery vehicle comprises: an expression vector encoding an Isl1 molecule, a recombinant viral vector encoding an Isl1 molecule, a replication-defective recombinant viral vector encoding an Isl1 molecule, a purified viral particle having a lower seroprevalence than a wild-type virus, a plasmid encoding an Isl1 molecule, a phage vector encoding an Isl1 molecule, lipids, liposomes, nanoparticles, a supercharged protein, a peptide, or any combination thereof. In other embodiments, the recombinant viral vector or the replication-defective recombinant viral vector comprises: lentivirus vectors, adenovirus vectors, adeno-associated virus (AAV) vectors, vesicular stomatitis virus (VSV) vectors, herpes simplex virus (HSV) vectors, vaccinia virus vectors, pox virus vectors, influenza virus vectors, respiratory syncytial virus vectors, parainfluenza virus vectors, foamy virus vectors, or retrovirus vectors.

In some embodiments, a virus particle comprises an Islet-1 (Isl1) nucleic acid sequence wherein the virus particle has a lower seroprevalence as compared to a wild-type virus. In certain embodiments, the virus particle comprises one or more ancestral capsid polypeptides. In some embodiments, the virus particle is an adeno-associated virus (AAV) comprising an AAV capsid polypeptide that exhibits a lower seroprevalence than does an AAV2, AAVs/Anc80 or AAV8 capsid polypeptide or a virus particle comprising an AAV2, AAVs/Anc80 or AAV8 capsid polypeptide.

In some embodiments, a composition comprises a virus particle comprising an Islet-1 (Isl1) nucleic acid sequence wherein the virus particle has a lower seroprevalence as compared to a wild-type virus. In certain embodiments, the virus particle comprises one or more ancestral capsid polypeptides. In certain embodiments, the composition comprises an Isl1 modulating agent, comprising small molecules, gene activating complexes, gene-editing complexes, oligonucleotides, siRNA, miRNA, RNAi, shRNA, peptides, antibodies, aptamers, enzymes or combinations thereof.

In other embodiments, a method of preventing, treating and/or reversing age-related hearing loss, noise-induced hearing loss or idiopathic hearing loss in a subject in need thereof, the method comprises administering to an outer and/or inner ear cell of the subject, a cationic liposome comprising a therapeutically effective amount of an Islet-1 (Isl1) nucleic acid sequence; a vector encoding an Islet-1 (Isl1) nucleic acid sequence; and/or an Isl1 molecule; and/or agents comprising small molecules that activate Isl1 in inner ear cells including hair cells.

In other embodiments, a method of preventing, treating and/or reversing age-related hearing loss, noise-induced hearing loss or idiopathic hearing loss in a subject in need thereof, the method comprising: administering to an outer and/or inner ear cell of the subject, a purified virus particle comprising an Islet-1 (Isl1) nucleic acid sequence wherein the Isl1 is overexpressed in the outer and/or inner ear cells as compared to expression of Isl1 in a normal outer or inner ear cell; thereby. The purified virus particle is an adeno-associated virus (AAV) comprising an AAV capsid polypeptide that exhibits a lower seroprevalence than does an AAV2, AAVs/Anc80 or AAV8 capsid polypeptide or a virus particle comprising an AAV2, AAVs/Anc80 or AAV8 capsid polypeptide. In some embodiments, the purified virus particle is Anc80 according to accession number GenBank: KT235804-KT235812.

Other aspects are described infra.

### BRIEF DESCRIPTION OF THE DRAWINGS

FIGS. 1A-1D show results using the AAV-Isl1 composition in gene therapy to treat deafness. FIG. 1A shows that AAV-Isl1 injection into postnatal inner ear results in robust Isl1 expression in both inner and outer hair cells in adult. FIG. 1B shows that two weeks after noise exposure (8-16 kHz, 100 dB, 2 hrs), significantly better hearing was maintained at the frequencies most vulnerable to noise damage (22 and 64 and 32 kHz) in the injected CBA/Caj ears shown by ABR and DPOAE. FIG. 1C shows that hearing was significantly better at 11.32 and 16 kHz in the injected DBA mice, 1 and 2 months after injection. FIG. 1D shows that significantly better hearing from 5.66 to 22.64 kHz was seen in the CD1 inner ear injected with AAV-Isl1, one and two months later. Significantly better DPOAE was also seen in CD1 injected with AAV-Isl1, indicating better outer hair cell function.

FIG. 2 shows that AAV2-GFP infects miniature pig inner hair cells two weeks after injection through round window membrane.

FIGS. 3A-3B show the long-term effect of hearing restoration by AAV-Isl1. FIG. 3A: Seven months after AAV-Isl1 injection into CD1 neonatal inner ear, ABR thresholds are significantly lower from low to mid frequencies in the injected than in uninjected control inner ears. In some frequencies (8 and 11.32 kHz) ABR threshold was even lower than at 3 month of age, a strong indication of hearing rescue that was sustained and improved over time. FIG. 3B: DPOAE from the same group of inner ears showed the significantly better threshold in the mid frequencies.

FIGS. 4A-4B show hearing restoration is Isl1 specific. AAV-GFP was used as control injection into neonatal CD1 inner ears with hearing studied one month later. 4A. The injected inner ear did not show any hearing improvement by ABR (4A) or DPOAE (4B), compared to the uninjected inner ears. Thus, hearing restoration by AAV-Isl1 is Isl1 gene specific.

FIG. 5 shows the long-term hearing rescue by AAV-Isl1 in age-related hearing loss (ARHL) mouse model. Ten months after AAV-Isl1 injection into CD1 neonatal mice by cochleostomy, auditory function (ABRs) and outer hair cell function (DPOAE) were significantly better in the injected ears compared to uninjected inner ears at 5.66 to 11.32 kHz. Combined the study demonstrated Isl1 mediated by AAV delivery significantly attenuates ARHL in different mouse models (CD1, DBA/2J and C57BL/6J) and protects against NIHL in CBA/CaJ.

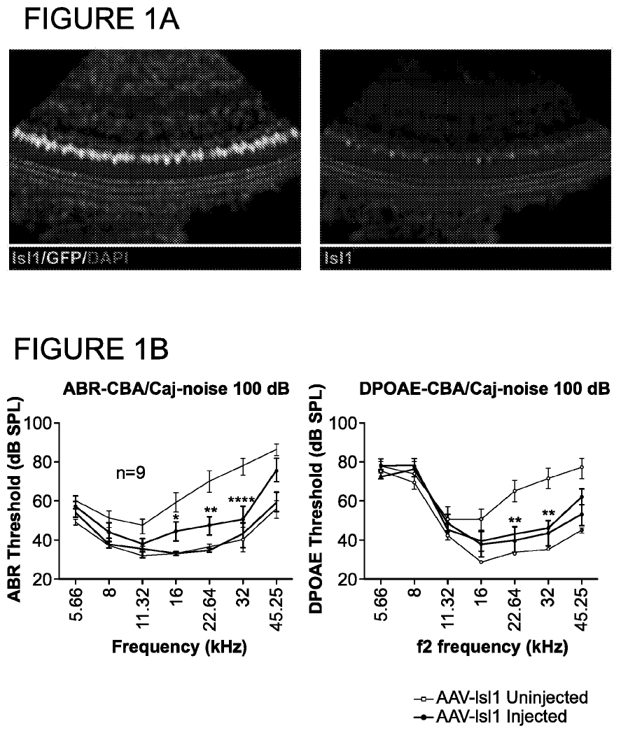

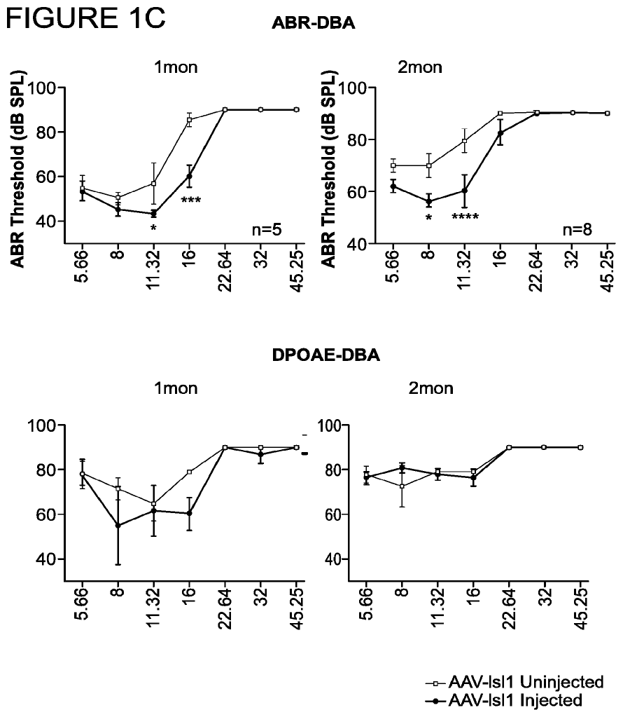

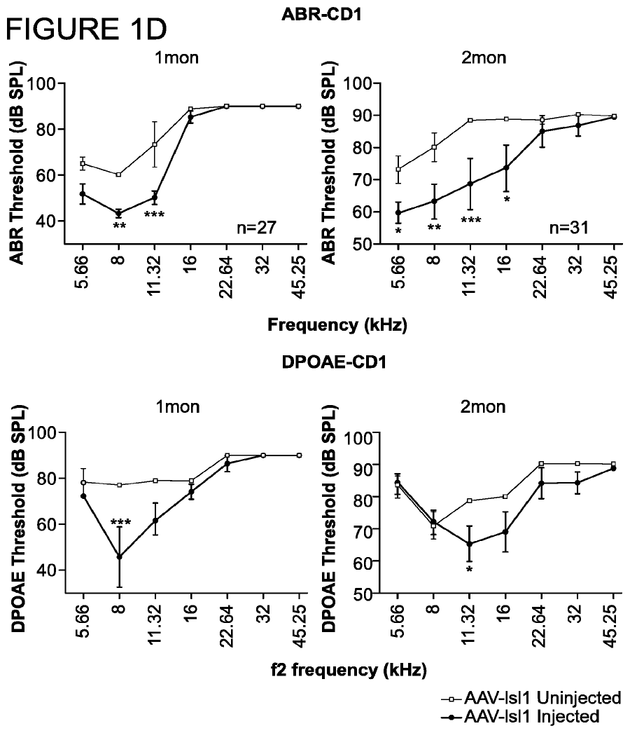

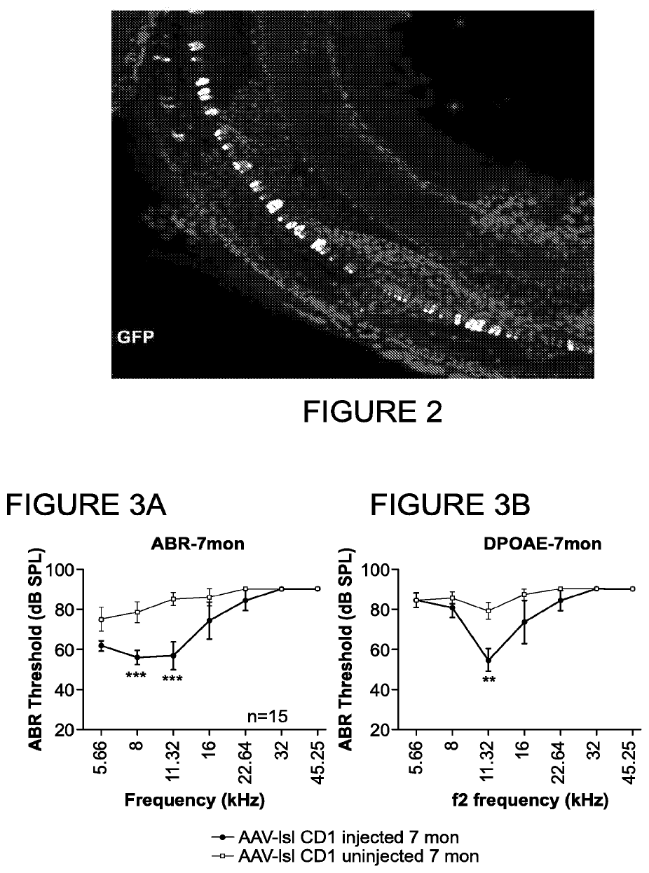

### DETAILED DESCRIPTION

Embodiments of the invention arc directed, inter alia, to viral vector mediated Islet-1 (Isl1) delivery into mouse inner ears in vivo to enable protection against, treatment and/or reversal of age-related hearing loss (ARHL), noise induced hearing loss (NIHL) and/or any idiopathic factors. In the examples section which follows, the results show that AAV mediated Islet-1 (Isl1) protection against hearing loss in multiple mouse strains with different types of ARHL and with noise exposure. This is the first time that a single gene overexpression in hair cells is sufficient to provide protection to a diverse group of mouse strains with ARHL and NIHL, which strongly supports that Isl1 overexpression may be a general mechanism that can be utilized to prevent, treat and reverse hearing loss in human.

Using a transgenic mouse model, it was shown that overexpression of Isl1 in hair cells protects against both ARHL and noise-induced hearing loss (NIHL). In aging transgenic mice that carry Cdh23 AHL allele (C57BL/6J-B6C3F1), hearing was maintained at 27-month of age; whereas in control littermates significant hearing loss started at 6-month of age that continued throughout aging. Further after noise exposure that induced permanent threshold shifts in control littermates, hearing loss in Isl1-hair cell overexpression mice were significantly attenuated. In both ARHL and NIHL models, Isl1 promotes hair cell survival (Huang, M., et al. J. Neurosci. 33, 15086-15094 (2013)). Thus, by enhancing hair cell survival through Isl1 in aging ear and after noise damage, or from idiopathic causes, hearing can be significantly improved.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, “Isl1” or “Isl1 molecules” refer to any and all Isl1-associated nucleic acid or protein sequences and includes any sequence that is orthologous or homologous to, or has significant sequence similarity to, an Isl1 nucleic acid or amino acid sequence derived from any animal including mammals (e.g., humans) and insects. The term also includes homologs, orthologs, mutants, variants or fragments thereof. Isl1 also includes all other synonyms that may be used to refer to this gene or the protein product of this gene (synonyms for this gene include ISL LIM homeobox 1, ISL1 transcription factor, LIM/homeodomain 2, ISL1 transcription factor, LIM/homeodomain, and islet-1).

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (e.g., derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “percent sequence identity” or having “a sequence identity” refers to the degree of identity between any given query sequence and a subject sequence.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, hinders, lubricants, gels, surfactants and the like, that may be used as media for a pharmaceutically acceptable substance.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids. As used herein, the terms “nucleic acid sequence”, “polynucleotide,” and “gene” are used interchangeably throughout the specification and include complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. The nucleic acid sequences, e.g. Isl1, may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The terms “polypeptide,” “peptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “seroprevalence” is understood in the art to refer to the proportion of subjects in a population that are seropositive (i.e., have been exposed to a particular pathogen or immunogen), and is calculated as the number of subjects in a population who produce an antibody against a particular pathogen or immunogen divided by the total number of individuals in the population examined. Immunoassays are well known in the art and include, without limitation, an immunodot, Western blot, enzyme immunoassays (EIA), enzyme-linked immunosorbent assay (ELISA), or radioimmunoassay (RJA).

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject. “Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

A “vector” is a composition of matter which comprises an isolated nucleic acid and which can be used to deliver the isolated nucleic acid to the interior of a cell. Examples of vectors include hut are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

### General Techniques

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, phage display, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (2002) Biochemistry (5th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2006) Biochemistry, 6^{th} Ed., W. H. Freeman Pub., New York, N.Y., all of which are herein incorporated in their entirety by reference for all purposes.

General methods in molecular and cellular biochemistry can be found in such standard textbooks as Molecular Cloning: A Laboratory Manual, 4th Ed. (Sambrook et al., Cold Spring Harbor Laboratory Press 2012); Short Protocols in Molecular Biology, 5th Ed. (Ausubel et al. eds., John Wiley & Sons 2002); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

### Inner Ear Cells

Sensory epithelia of the inner ear contain two major cell types: hair cells and supporting cells. (G. Wan et al., Semin Cell Dev Biol. 2013 May; 24(5): 448-459). Hair cells convert the energy in sound and head movements into neurophysiological signals that are relayed to the brainstem. In mammals, six sensory organs contain hair-cell epithelia. In the cochlear organ, which is specialized for hearing, hair cells reside within the organ of Corti, atop the basilar membrane, which vibrates in response to sound waves. Similarly, each of the five vestibular organs (the utricle, the saccule, and the three canal organs) contains sensory epithelia with hair cells that are activated by head movements and gravitational force. Hair cells are innervated by neurons whose cell bodies sit outside the sensory epithelium, either in a sensory ganglion within the temporal bone (afferent neurons) or in the hindbrain (efferent neurons).

There are two types of hair cells: outer and inner hair cells. Outer hair cells are distal from the spiral limbus, and generally there are three to five rows of hair cells that run the length of the cochlear duct (about 20,000 in number in humans). Inner hair cells are proximal to the spiral limbus. There is only one row of inner hair cells that run the length of the cochlear duct (about 3500 in number in humans).

The development, function, and maintenance of inner ear sensory epithelia are heavily dependent upon the supporting cells, which are non-sensory cells that reside between hair cells (Wan et al., supra). Unlike hair cells, which contact only the lumenal surface of the epithelium, supporting cells span the entire depth of the epithelium, from the basal lamina to the lumen. Supporting cells are linked to each other and to hair cells by tight and adherens junctions; and they communicate directly with other supporting cells by gap junctions. Within the mature sensory epithelia, supporting cells share many morphological and molecular features. For instance, all supporting cells in mammalian auditory and/or vestibular epithelia express the following genes at the protein and/or transcript level: Sox2, Sox9, Sox10, Jagged1, S100α, and p27^{kip1}. However, consistent with the large range of supporting cell functions, supporting cells in a given sensory epithelium show variation with respect to their shapes and molecular profiles. This is most pronounced in the mammalian organ of Corti, which has the greatest degree of supporting cell heterogeneity. Five different types of supporting cells are organized in rows along the organ's length. From the outer edge to the inner edge of the organ, they are: 1) Hensen's cells, 2) Deiters' cells, 3) pillar cells; 4) inner phalangeal cells; and 5) border cells. These supporting cells have distinct morphologies. Hensen's cells are cuboidal or slightly oblong. Inner phalangeal cells and border cells are columnar. The remaining cells—Deiters' and pillar cells—are architecturally exquisite cells, with a strong cytoskeleton, elongated processes, and large structural demands. For instance, the inner and outer pillar cells must maintain the structure of the tunnel of Corti, despite pressure from cells located on either side of the tunnel, during acoustic stimulation.

### Islet-1 (Isl1) Molecules

Islet-1 (Isl1) is a LIM-homeodomain transcription factor (LIM-HD) that is critical in the development and differentiation of the nervous system, such as the motor neurons. In addition, Isl1 controls pituitary and pancreas organogenesis, and is a key marker of cardiac progenitor cells. Functional studies using a conditional knockout model showed that Isl1 is also required for the development of retinal ganglion cells and forebrain cholinergic neurons.

Isl1 is expressed in the prosensory region of otocyst, and is subsequently expressed in early supporting cells and hair cells. Isl1 expression in hair cells is downregulated during later differentiation. In hair cells, expression of transcription factor of Pou4f3 leads to Lhx3 expression, which in turn suppresses Isl1 expression. This is confirmed by the lack of Lhx3 expression in the Pou4f3-null hair cells, and by overexpression of Lhx3 in cochlea nonsensory cells, which leads to Isl1 suppression.

Isl1 is known to be involved in motor neuron specification (Pfaff et al., Cell, 84(2):309-320 (1996)). Isl1 positive cells have also been identified in adult heart stem cells (Laugwitz et al., Development 135:193-205 (2008)).

The developmental role of Isl1 has also been reported. Isl1 is normally expressed in early inner ear development, suggesting a role in progenitor cell specification. Isl1 is not expressed in the cochlea, including auditory hair cells and supporting cells, in postnatal mice. In the postnatal utricle, Isl1 expression is expressed weakly in the supporting cells but not hair cells.

Isl1 polypeptides are, e.g., 349 amino acids in length and about 39 kDa. The chromosomal loci of Isl1 is 5q11.2. Human Isl1 sequences can be found in GenBank at Acc. No. NC_000005.9 (genomic), NT_006713.15 (genomic), NM_002202.2 (mRNA), and NP_002193.2 (protein). Antibodies that can be used to detect an Isl1 polypeptide are commercially available, e.g., from Cell Signaling Technology, Abcam, Novus Biologicals, Sigma-Aldrich, R&D Systems, Millipore, Abnova, and/or Invitrogen).

The studies herein demonstrate a general utility of Isl1 in attenuating ARHL of different origins through gene therapy by, for example, AAV-mediated Isl1 delivery into different postnatal mouse inbred strains with ARHL. The AAV vector (Anc80) targets inner and outer hair cells to deliver Isl1 to postnatal inner ears of DBA and CD1. DBA has Fascn2 mutation that gives rise to ARHL (Shin, J.-B., et al. J. Neurosci. 30, 9683-9694 (2010)), whereas the underlying gene mutations in ARHL of CD1 are unknown. In contrast to significant hearing loss in both strains starting at one month of age, AAV-Isl1 injected inner ear showed significant hearing protection. AAV-Isl1 injection into postnatal CBA/Caj inner ear was performed and hearing after noise exposure that causes permanent threshold shift (PTS) was evaluated. Significant protection was detected in the noise exposure model.

Accordingly, in embodiments, a composition comprises: an Isl1 gene, an Isl1 polynucleotide, an Isl1 oligonucleotide, an Isl1 protein, an Isl1 polypeptide, an Isl1 peptide, an Isl1 variant or mutants thereof (referred to collectively herein as Isl1 molecules). When administered to a target host cell in vitro or in vivo, the compositions comprising Isl1 molecules, increase the levels (e.g., protein levels) and/or activity (e.g., biological activity) of Isl1 in target host cells.

In some embodiments, a composition comprises a virus particle comprising an Islet-1 (Isl1) nucleic acid sequence wherein the virus particle has a lower seroprevalence as compared to a wild-type virus. In certain embodiments, the virus particle comprises one or more ancestral capsid polypeptides. In certain embodiments, the composition comprises an Isl1 modulating agent, comprising small molecules, gene activating complexes, gene-editing complexes, oligonucleotides, siRNA, miRNA, RNAi, shRNA, peptides, antibodies, aptamers, enzymes or combinations thereof.

In other embodiments, a method of preventing, treating and/or reversing age-related hearing loss, noise-induced hearing loss or idiopathic hearing loss in a subject in need thereof, the method comprises administering to an outer and/or inner ear cell of the subject, a vector encoding an Islet-1 (Isl1) nucleic acid sequence wherein the Isl1 is overexpressed in the outer and/or inner ear cells as compared to expression of Isl1 in a normal outer or inner ear cell. Examples of inner ear cells comprise: stria vascularis, hair cells, or supporting cells. Examples of vectors include, without limitation: lentivirus vectors, adenovirus vectors, adeno-associated virus (AAV) vectors, vesicular stomatitis virus (VSV) vectors, herpes simplex virus (HSV) vectors, vaccinia virus vectors, pox virus vectors, influenza virus vectors, respiratory syncytial virus vectors, parainfluenza virus vectors, foamy virus vectors, a retrovirus vector, recombinant viral vectors, eukaryotic vectors, naked DNA vectors, plasmids, or combinations thereof.

In certain embodiments, the Isl1 nucleic acid sequence is under control of a tissue specific promoter sequence wherein the promoter is a constitutive or inducible promoter.

In certain embodiments, the tissue specific promoter sequence is a hair-cell specific promoter sequence. Examples of hair cell specific promoter, include, without limitation: human cytomegalovirus (CMV) promoter, a chicken β-actin/CMV hybrid (CAG) promoter, or myosin VITA promoter.

In certain embodiments, the promoter is a support cell promoter. Examples of a support cell specific promoter, include, without limitation: a glial fibrillary acidic protein (GFAP) promoter, an excitatory amino acid transporter-1 (EAAT1) promoter, a glutamate transporter (GLAST) promoter or a murine cytomegalovirus (mCMV) promoter.

In some embodiments, the promoter is a ganglion neuron specific promoter sequence. Examples include: an ephrinB2, ephrinB3, trkB, trkc, GATA3, BF1, FGF10, FGF3, CSP, GFAP, or Islet1 promoter.

In one embodiment, the vector is an AAV vector. The vector can be modified to comprise capsid polypeptides having a lower seroprevalence in a subject as compared to a wild-type AAV vector.

In another embodiment, a method of expressing an exogenous Islet-1 (Isl1) nucleic acid sequence in an outer and/or inner ear cell in vitro or in vivo, comprises contacting the outer and/or inner ear cell with a delivery vehicle comprising an exogenous Islet-1 (Isl1) nucleic acid sequence wherein the Isl1 nucleic acid is overexpressed in the outer and/or inner ear cell as compared to expression of Isl1 in a normal cell. In embodiments, the delivery vehicle comprises: an expression vector encoding an Isl1 molecule, a recombinant viral vector encoding an Isl1 molecule, a replication-defective recombinant viral vector encoding an Isl1 molecule, a purified viral particle having a lower seroprevalence than a wild-type virus, a plasmid encoding an Isl1 molecule, a phage vector encoding an Isl1 molecule, lipids, liposomes, nanoparticles, a supercharged protein, a peptide, or any combination thereof. In other embodiments, the recombinant viral vector or the replication-defective recombinant viral vector comprises: lentivirus vectors, adenovirus vectors, adeno-associated virus (AAV) vectors, vesicular stomatitis virus (VSV) vectors, herpes simplex virus (HSV) vectors, vaccinia virus vectors, pox virus vectors, influenza virus vectors, respiratory syncytial virus vectors, parainfluenza virus vectors, foamy virus vectors, or retrovirus vectors.

In some embodiments, a virus particle comprises an Islet-1 (Isl1) nucleic acid sequence wherein the virus particle has a lower seroprevalence as compared to a wild-type virus. In certain embodiments, the virus particle comprises one or more ancestral capsid polypeptides. In some embodiments, the virus particle is an adeno-associated virus (AAV) comprising an AAV capsid polypeptide that exhibits a lower seroprevalence than does an AAV2, AAVs/Anc80 or AAV8 capsid polypeptide or a virus particle comprising an AAV2, AAVs/Anc80 or AAV8 capsid polypeptide. In certain embodiments, the wherein the purified virus particle is Anc80 according to accession number GenBank: KT235804-KT235812.

Isl1 Nucleic Acid Molecules: In some embodiments, the Isl1 molecule is an Isl1 gene, Isl1 polynucleotide, Isl1 oligonucleotide, mutants, orthologs, homologs, variants or combinations thereof. Any Isl1 gene or nucleic acid sequence can be expressed, e.g., in one or more auditory hair cells, using one or more expression constructs. Exemplary Isl1 nucleic acid sequences that may be usefully expressed include, but are not limited to, for example, nucleic acid sequences such as National Center for Biotechnology Information (NCBI) accession numbers NM_002202.2 (human Isl1 mRNA), BC031213.1 (human Isl1 cDNA), NM_021459.4 (murine Isl1 mRNA), BC132609.1 (murine Isl1 cDNA), and BC132263.1 (murine Isl1 cDNA), and any nucleic acid sequence with at least 50% (e.g., 60%, 70%, 80%, 85%, 90%, 95%, 98%, 99%, or 100%) sequence identity to NCBI accession numbers NM_002202.2, BC031213.1, NM_021459.4, BC132609.1, and BC132263.1. In some embodiments, Isl1 nucleic acids can include nucleic acids encoding an Isl1 polypeptide such as NCBI accession numbers EAW54861.1, NP_002193.2, P63171.1, NP_067434.3, AAI46164.1, AAI32264.1, ABM85672.1, EDM10395.1, ABM82484.1, EDL18368.1, and EDL18367.1, and any amino acid sequence with at least 50% (e.g., 60%, 70%, 80%, 85%, 90%, 95%, 98%, 99%, or 100%) sequence identity to NCBI accession numbers EAW54861.1, NP_002193.2, P63171.1, NP_067434.3, AAI46164.1, AAI32264.1, ABM85672.1, EDM10395.1, ABM82484.1, EDL18368.1, and EDL18367.1.

In some embodiments, DNA encoding Isl1 can be an unmodified wild type sequence. Alternatively, DNA encoding Isl1 can be modified using standard molecular biological techniques. For example, DNA encoding Isl1 can be altered or mutated, e.g., to increase the stability of the DNA or resulting polypeptide. Polypeptides resulting from such altered DNAs will retain the biological activity of wild type Isl1. In some embodiments, DNA encoding Isl1 can be altered to increase nuclear translocation of the resulting polypeptide. In some embodiments, DNA encoding Isl1 can be modified using standard molecular biological techniques to include an additional DNA sequence that can encode one or more of, e.g., detectable polypeptides, signal peptides, and protease cleavage sites.

In some embodiments, a composition comprises a vector encoding an Islet-1 (Isl1) nucleic acid sequence wherein the Isl1 nucleic acid sequence is under control of a tissue specific promoter sequence. In certain embodiments, the tissue specific promoter is a constitutive or inducible promoter. In one embodiment, the tissue specific promoter sequence is a hair-cell specific promoter sequence.

In certain embodiments, the vector comprises: a lentivirus vector, an adenovirus vector, an adeno-associated virus (AAV) vector, a vesicular stomatitis virus (VSV) vector, a herpes simplex virus (HSV) vector, a vaccinia virus vector, a pox virus vector, an influenza virus vector, a respiratory syncytial virus vector, a parainfluenza virus vector, a foamy virus vector, a retrovirus vector, a eukaryotic vector, or a plasmid.

In some embodiments of the invention, it may be desirable to use a cell, cell type, cell lineage or tissue specific expression control sequence to achieve cell type specific, lineage specific, or tissue specific expression of a desired polynucleotide sequence, for example, to express a particular nucleic acid encoding a polypeptide in only a subset of cell types, cell lineages, or tissues, or during specific stages of development. Illustrative examples of cell, cell type, cell lineage or tissue specific expression control sequences include, but are not limited to: an Atoh1 enhancer for all hair cells; a Pou4f3 promoter for all hair cells; a Myo7a promoter for all hair cells; an Hes5 promoter for vestibular supporting cells and cochlear inner phalangeal cells, Deiters cells and Pillar cells; and GFAP promoter for vestibular supporting cells and cochlear inner phalangeal cells, Deiters cells and Pillar cells; an ephrinB2, ephrinB3, trkB, trkc, GATA3, BF1, FGF10, FGF3, CSP, GFAP, or Islet1 promoter for ganglion neural cells.

Certain embodiments of the invention provide conditional expression of a polynucleotide of interest. For example, expression is controlled by subjecting a cell, tissue, organism, etc., to a treatment or condition that causes the polynucleotide to be expressed or that causes an increase or decrease in expression of the polynucleotide encoded by the polynucleotide of interest. Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al, 2003, Gene, 323:67), the cumate inducible gene switch (WO 2002/088346), tetracycline-dependent regulatory systems, etc.

Modified or Mutated Nucleic Acid Sequences: In some embodiments, any of the Isl1 nucleic acid sequences may be modified or derived from a native nucleic acid sequence, for example, by introduction of mutations, deletions, substitutions, modification of nucleobases, backbones and the like. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH₂—N(CH₃)—O—CH₂ [known as a methylene(methylimino) or MMI backbone], CH₂—O—N (CH₃)—CH₂, CH₂—N (CH₃)—N (CH₃)—CH₂ and O—N (CH₃)—CH₂—CH₂ backbones, wherein the native phosphodiester backbone is represented as O—P—O—CH). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

The isolated nucleic acid molecules of the present invention can be produced by standard techniques. For example, polymerase chain reaction (PCR) techniques can he used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

In some embodiments, the nucleic acids described herein, e.g., vectors, nucleic acids encoding an Isl1 polypeptide or active fragment thereof, or a nucleic acid encoding a protein that increases Isl1 expression, level, or activity, can be incorporated into a gene construct to be used as a part of a gene therapy protocol. The invention includes targeted expression vectors for in vivo transfection and expression of a polynucleotide that encodes an Isl1 polypeptide or active fragment thereof, or a protein that increases Isl1 expression, level, or activity as described herein, in particular cell types (e.g., auditory hair cells or cells with, or that are capable of differentiating into a cell with, one or more characteristics of an auditory hair cell). Such expression constructs can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, poxvirus, alphavirus, and herpes simplex virus-1, or recombinant bacterial or eukaryotic plasmids. Viral vectors transfect cells directly; plasmid DNA can be delivered naked or with the help of, for example, cationic liposomes (e.g., LIPOFECTAMINE) or derivatized (e.g., antibody conjugated), polylysine conjugates, gramicidin S, artificial viral envelopes or other such intracellular carriers, as well as direct injection of the gene construct or CaPO4 precipitation carried out in vivo.

Isl1 Polypeptides and Proteins: In some embodiments, the Isl1 molecule is an Isl1 polypeptide. Exemplary useful Isl1 polypeptides include, but are not limited to, for example, GenBank Acc. Nos. EAW54861.1, NP_002193.2, P63171.1, NP_067434.3, AAI46164.1, AAI32264.1, ABM85672.1, EDM10395.1, ABM82484.1, EDL18368.1, and EDL18367.1, and any amino acid sequence with at least 50% (e.g., 60%, 70%, 80%, 85%, 90%, 95%, 98%, 99%, or 100%) sequence identity to NCBI accession numbers EAW54861.1, NP_002193.2, P63171.1, NP_067434.3, AAI46164.1, AAI32264.1, ABM85672.1, EDM10395.1, ABM82484.1, EDL18368.1, and EDL18367.1.

Isl1 polypeptides can be generated using recombinant techniques or using chemical synthesis. Methods for generating such polypeptides, and methods required for the purification of such polypeptides, are known in the art, see, e.g., Sambrook, Molecular Cloning: A Laboratory Manual (CSHL Press, 3^{rd} Edition, 2001).

Modifications can be made to a protein to alter the pharmacokinetic properties of the protein to make it more suitable for use in protein therapy. For example, such modifications can result in longer circulatory half-life, an increase in cellular uptake, improved distribution to targeted tissues, a decrease in clearance and/or a decrease of immunogenicity. A number of approaches useful to optimize the therapeutic activity of a protein, e.g., a therapeutic protein described herein, e.g., a Isl1 modulating agent, a Isl1 polypeptide, peptide or peptide mimetic, a Isl1 analog are known in the art, including chemical modification.

In some embodiments, the Isl1 molecule includes a cell-penetrating peptide sequence that facilitates delivery of Isl1 to the intracellular space, e.g., HIV-derived TAT peptide, penetratins, transportans, or hCT derived cell-penetrating peptides, see, e.g., Caron et al., Mol Ther. 3:310-8, 2001; Langel, Cell-Penetrating Peptides: Processes and Applications (CRC Press, Boca Raton Fla. 2002); El-Andaloussi et al., Curr. Pharm. Des., 11:3597-611, 2005; and Deshayes et al., Cell. Mal. Life Sci., 62:1839-49, 2005.

### Delivery Vehicles

Delivery vehicles as used herein, include any types of molecules for delivery of the compositions embodied herein, both for in vitro or in vivo delivery. Examples, include, without limitation: expression vectors, nanoparticles, colloidal compositions, lipids, cationic lipids, liposomes, nanosomes, carbohydrates, peptides, supercharged proteins or peptides, organic or inorganic compositions and the like.

In one embodiment, a method for in vivo introduction of nucleic acid into a cell comprises a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

In some embodiments, a delivery vehicle is a vector. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

In certain embodiments, vectors comprise a selection gene, also termed a selectable marker. Typical selection genes encode proteins that (a) confer resistance to antibiotics or other toxins, e.g., ampicillin, neomycin, hygromycin, methotrexate, Zeocin, Blastocidin, or tetracycline, (b) complement auxotrophic deficiencies, or (c) supply critical nutrients not available from complex media, e.g., the gene encoding D-alanine racemase for Bacilli. Any number of selection systems may be used to recover transformed cell lines. These include, but are not limited to, the herpes simplex virus thymidine kinase (Wigler et al, (1977) Cell, 11:223-232) and adenine phosphoribosyltransferase (Lowy et al, (1990) Cell, 22:817-823) genes which can be employed in tk- or aprt-cells, respectively.

In certain embodiments, the Isl1 molecules in a vector are under the control of a tissue specific, constitutive, or inducible promoter. The promoter can be any desired promoter, selected by known considerations, such as the level of expression of the DNA operatively linked to the promoter and the cell type in which the DNA is to be expressed, e.g., hair cells or support cells. Promoters can be an exogenous or an endogenous promoter. Promoters can be prokaryotic, eukaryotic, fungal, nuclear, mitochondrial, viral, etc. Additionally, chimeric regulatory promoters for targeted gene expression can be utilized. In certain embodiments, promoters comprise cochlear hair specific promoters or support cell specific promoters.

Examples of other promoters include: the MYO7A promoter, which exhibits strong, selective expression in hair cells of the cochlea and vestibule; the glial fibrillary acid protein (GFAP) promoter was shown to have selective activity within certain subpopulations of support cells; the murine CMV (mCMV) promoter, which exhibits selectivity for astrocytes; the astrocytic glutamate transporter (GLAST) is expressed only in border cells and inner phalangeal cells of mature guinea pigs and in P0 cochlear explants culture; the Jagged-1 and Notch 1, which may also be useful for support cell specific expression.

Examples of support cell specific promoters include: the glial fibrillary acidic protein (GFAP) promoter, the excitatory amino acid transporter-1 (EAAT1) promoter, the GLAST promoter and the murine cytomegalovirus (mCMV) promoter. Examples of preferred hair cell specific promoters include: the human cytomegalovirus (CMV) promoter, the chicken β-actin/CMV hybrid (CAG) promoter, and the myosin VIIA promoter. In one embodiment, the preferred promoter is the CAG promoter.

Examples of ganglion neuron cell promoters include: an ephrinB2, ephrinB3, trkB, trkc, GATA3, BF1, FGF10, FGF3, CSP, GFAP, or Islet1 promoter.

In certain embodiments, the tissue specific promoter sequence is a hair-cell specific promoter sequence. Examples of hair cell specific promoter, include, without limitation: human cytomegalovirus (CMV) promoter, a chicken β-actin/CMV hybrid (CAG) promoter, or myosin VIIA promoter.

In certain embodiments, the promoter is a support cell promoter. Examples of a support cell specific promoter, include, without limitation: a glial fibrillary acidic protein (GFAP) promoter, an excitatory amino acid transporter-1 (EAAT1) promoter, a glutamate transporter (GLAST) promoter or a murine cytomegalovirus (mCMV) promoter.

Additional expression vectors also can include, for example, segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Vectors include, for example, viral vectors (such as adenoviruses (Ad), AAV, lentivirus, vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

In one embodiment, the delivery vehicle is a virus vector comprising a capsid polypeptide exhibiting a lower seroprevalence than a wild-type virus. Examples include any virus vector, such as, adeno-associated virus (AAV) comprising ancestral AAV capsid polypeptides e.g. Anc80. AAV vector Anc80 (accession number GenBank: KT235804-KT235812), is an ancestor of AAV 1, 2, 8 and 9. See, for example, Zinn, E. et al., 2015, Cell Reports 12:1056-1068 and Vandenberghe, L. H et al., PCT/US2014/060163, both of which are incorporated by reference herein, in their entirety.

In other embodiments, the delivery vehicle is an ancestral virus particle. In some embodiments, a virus particle comprises an Islet-1 (Isl1) nucleic acid sequence wherein the virus particle has a lower seroprevalence as compared to a wild-type virus. In certain embodiments, the virus particle comprises one or more ancestral capsid polypeptides. In some embodiments, the virus particle is an adeno-associated virus (AAV) comprising an AAV capsid polypeptide that exhibits a lower seroprevalence than does an AAV2, AAVs/Anc80 or AAV8 capsid polypeptide or a virus particle comprising an AAV2, AAVs/Anc80 or AAV8 capsid polypeptide. In certain embodiments, the wherein the purified virus particle is Anc80 according to accession number GenBank: KT235804-KT235812.

Virus particles assembled from predicted ancestral viral sequences can exhibit less, sometimes significantly less, seroprevalence than current-day, contemporary virus particles. As indicated herein, ancestral virus particles exhibit less seroprevalence than do contemporary virus particles (i.e., virus particles assembled using contemporary virus sequences or portions thereof). Simply by way of example, see Xu et al. (2007, Am. J. Obstet. Gynecol., 196:43.el-6); Paul et al. (1994, J. Infect. Dis., 169:801-6); Sauerbrei et al. (2011), Eurosurv., 16(44):3); and Sakhria et al. (2013, PLoS Negl. Trop. Dis., 7:e2429), each of which determined seroprevalence for a particular antibody in a given population.

Ancestral virus particles comprise ancestral virus sequences. After a predicted ancestral sequence of a virus or portion thereof has been obtained (see, for example, Vandenberghe, L. H et al., PCT/US2014/060163), the actual nucleic acid molecule and/or polypeptide(s) can be generated, e.g., synthesized. Methods of generating an artificial nucleic acid molecule or polypeptide based on a sequence obtained, for example, in silico, are known in the art and include, for example, chemical synthesis or recombinant cloning. Additional methods for generating nucleic acid molecules or polypeptides are known in the art.

Once an ancestral polypeptide has been produced, or once an ancestral nucleic acid molecule has been generated and expressed to produce an ancestral polypeptide, the ancestral polypeptide can be assembled into an ancestral virus particle using, for example, a packaging host cell. The components of a virus particle (e.g., rep sequences, cap sequences, inverted terminal repeat (ITR) sequences) can be introduced, transiently or stably, into a packaging host cell using one or more vectors as described herein. One or more of the components of a virus particle can be based on a predicted ancestral sequence as described herein, while the remaining components can be based on contemporary sequences. In some instances, the entire virus particle can he based on predicted ancestral sequences. Ancestral virus particles can be purified using routine methods. As used herein, “purified” virus particles refer to virus particles that are removed from components, in the mixture in which they were made such as, but not limited to, viral components (e.g., rep sequences, cap sequences), packaging host cells, and partially- or incompletely-assembled virus particles.

With respect to, for example, ancestral AAV capsid polypeptides, the seroprevalence and/or extent of neutralization can be compared, for example, to an AAV8 capsid polypeptide or virus particle that includes an AAV8 capsid polypeptide, or an AAV2 capsid polypeptide or virus particle that includes an AAV2 capsid polypeptide. It is generally understood in the art that AAV8 capsid polypeptides or virus particles exhibit a seroprevalence, and a resulting neutralization, in the human population that is considered low, while AAV2 capsid polypeptide or virus particles exhibit a seroprevalence, and a resulting neutralization, in the human population that is considered high. The particular seroprevalence will depend upon the population examined as well as the immunological methods used, but there are reports that AAV8 exhibits a seroprevalence of about 22% up to about 38%, while AAV2 exhibits a seroprevalence of about 43.5% up to about 72%. See, for example, Boutin et al., 2010, “Prevalence of serum IgG and neutralizing factors against AAV types 1, 2, 5, 6, 8 and 9 in the healthy population: implications for gene therapy using AAV vectors,” Hum. Gene Ther., 21:704-12. See, also Calcedo et al., 2009, J. Infect. Dis., 199:381-90.

Thus, ancestral virus sequences are suitable for use in vectors or vector systems for gene transfer. To predict an ancestral viral sequence, nucleotide or amino acid sequences first are compiled from a plurality of contemporary viruses or portions thereof. Viruses include, without limitation, adeno-associated virus (AAV), adenovirus (AV), human immunodeficiency virus (HIV), retrovirus, lentivirus, herpes simplex virus (HSV), measles, vaccinia virus, pox virus, influenza virus, respiratory syncytial virus, parainfluenza virus, foamy virus, or any other virus to which pre-existing immunity is considered a problem. Sequences from as few as two contemporary viruses or portions thereof can be used, however, it is understood that a larger number of sequences of contemporary viruses or portions thereof is desirable so as to include as much of the landscape of modern day sequence diversity as possible, but also because a larger number of sequences can increase the predictive capabilities of the algorithms described and used. Such sequences can be obtained, for example, from any number of public databases including, without limitation, GenBank, UniProt, EMBL, International Nucleotide Sequence Database Collaboration (INSDC), or European Nucleotide Archive. Additionally, or alternatively, such sequences can be obtained from a database that is specific to a particular organism (e.g., HIV database). The contemporary sequences can correspond to the entire genome, or only a portion of the genome can be used such as, without limitation, sequences that encode one or more components of the viral capsid, the replication protein, or the ITR sequences.

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (Reviewed in Hu and Pathak, Pharmacol. Rev. 52: 493-511 (2000); Young et al., J. Pathol. 208:229-318 (2006)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel et al. (eds.), 2002, “Short Protocols in Molecular Biology,” John Wiley & Sons, Inc., and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include Crip, Cre, 2, Am, pA12 and PA317 (For a review, see Miller et. al, Hum. Gene Ther. 1:5-14 (1990)). Retroviruses have been used to introduce a variety of genes into many different cell types, including epithelial cells, in vitro and/or in vivo (see, for example Eglitis et al., Science 230:1395-1398 (1985); Danos and Mulligan, Proc. Natl. Acad. Sci. USA 85:6460-6464 (1988); Wilson et al., Proc. Natl. Acad. Sci. USA 85:3014-3018 (1988); Armentano et al., Proc. Natl. Acad. Sci. USA 87:6141-6145 (1990); Miller et al., Blood 76:271-8 (1990); Huber et al. Proc. Natl. Acad. Sci. USA 88:8039-8043 (1991); Ferry et al. Proc. Natl. Acad. Sci. USA 88:8377-8381 (1991); Chowdhury et al. Science 254:1802-1805 (1991); van Beusechem et al. Proc. Natl. Acad. Sci. USA 89:7640-7644 (1992); Kay et al. Human Gene Therapy 3:641-647 (1992); Dai et al. Proc. Natl. Acad. Sci. USA 89:10892-10895 (1992); Hwu et al. J. Immunol. 150:4104-4115 (1993); Cavazzana-Calvo et al., Science 288:669-672 (2000); U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The generation of replication-deficient adenovirus was achieved through the manipulation of the genome of an adenovirus, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases (kb)) relative to other gene delivery vectors (Berkner et al., supra; Haj-Ahmand and Graham, J. Viral. 57:267 (1986). Additionally, special high-capacity adenoviral (HC-Ad) vectors have been created that can contain more than 30 kb of transgene (Kochanek et al., Hum. Gene Ther. 10:2451-9 (1999)).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle (Reviewed in McCarty et al., Annu Rev Genet 38:819-45 (2004); Daya et al., Clin. Microbial. Rev. 21: 583-93 (2008)). It is also one of the few viruses that may integrate its DNA into non-dividing cells, and exhibits a high frequency of stable integration that can lead to long term expression (see, for example Samulski et al., J. Viral. 63:3822-3828 (1989); and McLaughlin et al., J. Viral. 62:1963-1973 (1989); Flotte et al., Am. J. Respir. Cell. Mol. Biol. 7:349-356 (1992); Miller et al., Nature Genet. 36:767-773 (2004)). Vectors containing as little as 300 base pairs of AAV can be packaged and can integrate. Space for exogenous DNA is limited to about 4 kb. An AAV vector such as that described in Tratschin et al., Mol. Cell. Biol. 5:3251-3260 (1985) can be used to introduce DNA into cells. Through the use of AAV vectors, which are derived from many different serotypes, a variety of nucleic acids have been introduced into different cell types (see, for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flottc et al., J. Biol. Chem. 268:3781-3790 (1993); Summerford et al., J. Virol. 72:1438-45 (1998); Davidson et al., Proc. Natl. Acad. Sci. USA 97:3428-32 (2000); Zabner et al., J. Virol. 74:3852-8 (2000); Rabinowitz J E et al., J. Virol. 76:791-801 (2002); Davidoff et al., Mol Ther. 11:875-88 (2005); Mueller et al., Gene Ther. 15:858-63. (2008)).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See, for example, Chen et al, BioTechniques, 34: 167-171 (2003), which is incorporated herein, by reference, in its entirety.

Several delivery methods may be utilized in conjunction with the isolated nucleic acid sequences for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). An example of a non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

The nucleic acid sequences of the invention can also he delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In addition to viral transfer methods, such as those illustrated above, non-viral methods can also be employed to cause expression of a nucleic acid compound described herein (e.g., a polypeptide encoding Isl1 nucleic acid or a polypeptide encoding a compound that increases Isl1 expression levels, or activity) in the tissue of a subject (For a review, see Niidome et al., Gene Ther. 9:1647-52 (2002)). Typically, non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-cationic conjugates such as polyamine and polylysine, and artificial viral envelopes. Other embodiments include plasmid injection systems such as are described in Cohen et al., Gene Ther. 7:1896-905 (2000); Tam et al., Gene Ther. 7:1867-74 (2000); Meuli et al., J. Invest. Dermatol. 116:131-135 (2001); or Fenske et al., Methods Enzymol. 346:36-71 (2002).

In some embodiments, a gene encoding Isl1 molecule is entrapped in liposomes bearing positive charges on their surface (e.g., lipofectins), which can be tagged with an adaptor molecule, such as biotin or antibodies against cell surface antigens of the target tissue, to facilitate targeting (Bartlett et al., Nat. Biotechnol. 17:181-6 (1999); Arnold et al., Mol. Ther. 14:97-106 (2006); PCT publication WO91/06309; Japanese patent application 1047381; and European patent publication EP-A-43075).

In clinical settings, the gene delivery systems for the therapeutic gene can be introduced into a subject by any of a number of methods, each of which is familiar in the art or is described herein. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

In some aspects, Isl1 can be expressed using expression constructs, e.g., naked DNA constructs, DNA vector based constructs, and/or viral vector and/or viral based constructs.

Naked DNA constructs and the therapeutic use of such constructs are well known to those of skill in the art (see, e.g., Chiarella et al., Recent Patents Anti-Infect. Drug Disc., 3:93-101, 2008; Gray et al., Expert Opitz. Biol. Ther., 8:911-922, 2008; Melman et al., Hum. Gene Ther., 17:1165-1176, 2008). In some embodiments, naked DNA constructs include one or more therapeutic nucleic acids (e.g., DNA encoding Isl) and a promoter sequence. A naked DNA construct can be a DNA vector, commonly referred to as pDNA. Naked DNA typically do not incorporate into chromosomal DNA. Generally, naked DNA constructs do not require, or are not used in conjunction with, the presence of lipids, polymers, or viral proteins. Such constructs may also include one or more of the non-therapeutic components described herein.

DNA vectors are known in the art and typically are circular double stranded DNA molecules. DNA vectors usually range in size from three to five kilo-base pairs (e.g., including inserted therapeutic nucleic acids). Like naked DNA, DNA vectors can be used to deliver and express one or more therapeutic proteins in target cells. DNA vectors do not incorporate into chromosomal DNA.

Generally, DNA vectors include at least one promoter sequence that allows for replication in a target cell. Uptake of a DNA vector may be facilitated (e.g., improved) by combining the DNA vector with, for example, a cationic lipid, and forming a DNA complex.

In some embodiments, DNA vectors can be introduced into target cells via conventional transformation or transfection techniques. As used herein, the terms “transformation” and “transfection” are intended to refer to a variety of art-recognized techniques for introducing foreign nucleic acid (e.g., DNA) into a target cell, including calcium phosphate or calcium chloride co-precipitation, DEAE-dextran-mediated transfection, lipofection, or electroporation.

The present application also provides such expression constructs formulated as a pharmaceutical composition, e.g., for administration to a subject. Such pharmaceutical compositions are not limited to one expression construct and rather can include two or more expression constructs (e.g., two, three, four, five, six, seven, eight, nine, ten or more expression constructs).

All the molecular biological techniques required to generate an expression construct described herein are standard techniques that will be appreciated by one of skill in the art. Detailed methods may also be found, e.g., Current Protocols in Molecular Biology, Ausubel et al. (eds.) Greene Publishing Associates, (1989), Sections 9.10 9.14 and other standard laboratory manuals. DNA encoding altered Isl1 can be generated using, e.g., site directed mutagenesis techniques.

For recombinant proteins, the choice of expression system can influence pharmacokinetic characteristics. Differences in post-translational processing between expression systems can lead to recombinant proteins of varying molecular size and charge, which can affect circulatory half-life, rate of clearance and immunogenicity, for example. The pharmacokinetic properties of the protein may be optimized by the appropriate selection of an expression system, such as selection of a bacterial, viral, or mammalian expression system. Exemplary mammalian cell lines useful in expression systems for therapeutic proteins are Chinese hamster ovary, (CHO) cells, the monkey COS-1 cell line and the CV-1 cell line.

The recombinant expression vectors of the invention can be designed for expression of Isl1 polypeptides in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, Gene Expression Technology: Methods in Enzymology, 185, (Academic Press, San Diego, Calif. 1990). Alternatively, the recombinant expression vector can be transcribed and translated in vitro, for example using T7 promoter regulatory sequences and T7 polymerase.

### Routes of Administration

The route of administration will vary depending on the disease being treated. Hair cell loss and vestibular disorders can be treated using direct therapy using systemic administration and/or local administration. In some embodiments, the route of administration can be determined by a subject's health care provider or clinician, for example following an evaluation of the subject.

In some embodiments, compositions comprising one or more Isl1 molecules can be administered to a subject, e.g., a subject identified as being in need of treatment using a systemic route of administration. Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

Alternatively, or in addition, one or more Isl1 molecules can be administered to a subject, e.g., a subject identified as being in need of treatment for hair cell loss, using a local route of administration. Such local routes of administration include administering one or more compounds into the ear of a subject and/or the inner ear of a subject, for example, by injection and/or using a pump.

In some embodiments, one or more Isl1 molecules embodied herein (e.g. vectors encoding Isl1, Isl1 polynucleotides, Isl1 oligonucleotides, Isl1 polypeptides, Isl1 peptides, or combinations thereof, etc.) can be injected into the ear (e.g., auricular administration), such as into the luminae of the cochlea (e.g., the Scala media, Sc vestibulae, and Sc tympani). For example, one or more Isl1 molecules can be administered by intratympanic injection (e.g., into the middle ear), and/or injections into the outer, middle, and/or inner ear. Such methods are routinely used in the art, for example, for the administration of steroids and antibiotics into human ears. Injection can be, for example, through the round window of the ear or through the cochlear capsule.

In some embodiments, the modes of administration described above may be combined in any order and can be simultaneous or interspersed.

### Pharmaceutical Compositions

In some embodiments, one or more Isl1 molecules or vectors encoding one or more Isl1 molecules, can be formulated as a pharmaceutical composition. Pharmaceutical compositions containing one or more Isl1 molecules can be formulated according to the intended method of administration.

One or more Isl1 molecules or vectors encoding one or more Isl1 molecules, can be formulated as pharmaceutical compositions for direct administration to a subject. Pharmaceutical compositions containing one or more compounds can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In some embodiments, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral.

A pharmaceutical composition can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa., 1990.

Alternatively or in addition, the pharmaceutical compositions can be formulated for systemic parenteral administration by injection, for example, by bolus injection or continuous infusion. Such formulations can be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

In addition to the formulations described previously, the compositions can also be formulated as a depot preparation. Such long acting formulations can be administered by implantation (e.g., subcutaneously). Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions formulated for systemic oral administration can take the form of tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

In some embodiments, the pharmaceutical compositions described herein can include one or more of the Isl1 molecules formulated according to any of the methods described above, and one or more cells obtained to the methods described herein.

### Methods of Treatment

In some embodiments, the treatment, inclusive of prevention and reversal, of hearing loss or auditory hair cell loss includes steps whereby compositions comprising one or more Isl1 molecules, e.g. a vector encoding an Isl1 molecule, are administered to a subject. This method of treatment is referred to as direct therapy. Loss of synapses between hair cells and auditory neurons are known to be involved in NIHL and ARHL. Treatment of Isl1 can also be used to maintain or increase the number of synapses, and reverse the loss so hearing loss can be reduced or revised.

In some embodiments, the treatment of outer, inner and/or auditory hair cell loss includes steps whereby one or more target cells are contacted, e.g., in vitro, with compositions comprising one or more one or more Isl1 molecules, and are then administered to the ear (e.g., the inner ear) of the subject. This method of therapy is referred to as cell therapy.

In some embodiments, the methods include steps whereby one or more target cells that have been contacted with one or more compositions comprising one or more one or more Isl1 molecules are administered to the ear (e.g., inner ear) of a subject in combination with one or more Isl1 modulating compounds or any other therapeutic agent. This method of treatment is referred to as combination therapy.

In some embodiments, a method of preventing, treating and/or reversing age-related hearing loss, noise-induced hearing loss or idiopathic hearing loss in a subject in need thereof, the method comprises administering to an outer and/or inner ear cell of the subject, a virus vector comprising an Islet-1 (Isl1) nucleic acid sequence wherein the Isl1 is overexpressed in the outer and/or inner ear cells as compared to expression of Isl1 in a normal outer or inner ear cell; and/or, administering cells comprising a vector encoding an Islet-1 (Isl1) nucleic acid sequence; and/or an Isl1 molecule; and/or agents comprising small molecules that activate Isl1 in inner ear cells including hair cells. Inner ear cells comprise: stria vascularis, hair cells, supporting cells or ganglion neurons.

In some embodiments, the Isl1 nucleic acid sequence is under control of a tissue specific promoter sequence wherein the promoter is a constitutive or inducible promoter. The tissue specific promoter sequence is a hair-cell specific promoter sequence, a stria vascularis specific promoter sequence or a supporting cell specific promoter sequence or a ganglion neuron specific promoter sequence.

Where appropriate, following treatment, the human can be tested for an improvement in hearing or in other symptoms related to inner ear disorders. Methods for measuring hearing arc well-known and include pure tone audiometry, air conduction, and bone conduction tests. These exams measure the limits of loudness (intensity) and pitch (frequency) that a human can hear. Hearing tests in humans include behavioral observation audiometry (for infants to seven months), visual reinforcement orientation audiometry (for children 7 months to 3 years) and play audiometry for children older than 3 years. Oto-acoustic emission testing can be used to test the functioning of the cochlear hair cells, and electro-cochleography provides information about the functioning of the cochlea and the first part of the nerve pathway to the brain. In some embodiments, treatment can be continued with or without modification or can be stopped.

Cell Therapy: In some embodiments, one or more Isl1 molecules or vectors encoding one or more Isl1 molecules, can be used to treat a cell in vitro (e.g., an auditory hair cell or a cell with, or that is capable of acquiring, one or more characteristics of an auditory hair cell, a stem cell etc). Such cells can then be transplanted or implanted into a subject in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Accordingly, in certain embodiments, protection against or treatment for hearing loss in a subject in need thereof, comprises administering to an outer and/or inner ear cell of the subject, a vector encoding an Islet-1 (Isl1) nucleic acid sequence wherein the Isl1 is overexpressed in the outer and/or inner ear cells as compared to expression of Isl1 in a normal outer or inner ear cell; and/or, administering cells comprising a vector encoding an Islet-1 (Isl1) nucleic acid sequence; and/or an Isl1 molecule; and/or Isl1 activating agents comprising small molecules.

Implantation Methods: In some embodiments, cells contacted in vitro with one or more compositions comprising Isl1 molecules can be transplanted or implanted, such as in the form of a cell suspension, into the ear by injection, such as into the luminae of the cochlea. Injection can be, for example, through the round window of the ear or through the bony capsule surrounding the cochlea. The cells can he injected through the round window into the auditory nerve trunk in the internal auditory meatus or into the scala tympani.

In some embodiments, the cells described herein can be used in a cochlear implant, for example, as described in Edge et al. (U.S. Publication No. 2007/0093878).

Combination Therapies: In some embodiments, the present invention provides methods for treating a subject with one or more compounds using the direct administration and cell therapy methods described above. For example, a composition comprising an Isl1 molecule can be administered with a second therapeutic, such as a therapeutic that may affect a hearing disorder. Such ototoxic drugs include the antibiotics neomycin, kanamycin, amikacin, viomycin, gentamycin, tobramycin, erythromycin, vancomycin, and streptomycin; chemotherapeutics such as cisplatin; nonsteroidal anti-inflammatory drugs (NSAIDs) such as choline magnesium trisalicylate, diclofenac, diflunisal, fenoprofen, flurbiprofen, ibuprofen, indomethacin, ketoprofen, meclofenamate, nabumetone, naproxen, oxaprozin, phenylbutazone, piroxicam, salsalate, sulindac, and tolmetin; diuretics; salicylates such as aspirin; and certain malaria treatments such as quinine and chloroquine. For example, a human undergoing chemotherapy can be treated using compounds and methods described herein. The chemotherapeutic agent cisplatin, for example, is known to cause hearing loss. Therefore, a composition comprising one or more Isl1 molecules can be administered with cisplatin therapy to prevent or lessen the severity of the cisplatin side effect. Such a composition can be administered before, after and/or simultaneously with the second therapeutic agent. The two or more agents can be administered by different routes of administration.

In some embodiments, the compositions embodied herein can be administered with an Isl1 modulating agent. An Isl1 modulating agent can be any molecule that can modulate the expression, function, activity of Isl1 in vitro or in vivo as compared to a normal baseline of Isl1 expression, function or activity. Examples of Isl1 modulating agents, include, without limitation: antibodies, (polyclonal or monoclonal), neutralizing antibodies, antigen-binding antibody fragments, peptides, proteins, peptide-mimetics, aptamers, oligonucleotides, enzymes, hormones, small molecules, nucleic acids, nucleic acid analogues, carbohydrates, or variants thereof, transcriptional activators, promoters, enhancers, oligonucleotides, inhibitors of repressors, pseudo-complementary-PNA (pcPNA), microRNA, siRNA shRNA, miRNA, antisense oligonucleic acids (ODNs), locked nucleic acids (LNA), peptide nucleic acids (PNA), DNA or nucleic acid analogues. In some embodiments, nucleic acids are nucleic acid analogues, for example but not limited to peptide nucleic acid (PNA), pseudo-complementary PNA (pcPNA), inhibitors or suppressors of the wnt/β-catenin pathway, locked nucleic acid (LNA), gene editing complexes (e.g. CRISPR/Cas) and analogues thereof.

A nucleic acid may be single or double stranded, and can be selected from a group comprising; nucleic acid encoding a protein of interest, oligonucleotides, PNA, etc. Such nucleic acid sequences include, but are not limited to nucleic acid sequence encoding proteins that act as transcriptional repressors, antisense molecules, ribozymes, small inhibitory nucleic acid sequences (including, but not limited to RNAi, shRNAi, siRNA, micro RNAi (mRNAi)) and antisense oligonucleotides, etc. A protein and/or peptide inhibitor or fragment thereof, can include, but is not limited to mutated proteins; therapeutic proteins and recombinant proteins. Proteins and peptide inhibitors can also include, for example, genetically modified proteins and peptides, synthetic peptides, chimeric proteins, antibodies, humanized proteins, humanized antibodies, chimeric antibodies, monoclonal and polyclonal antibodies, modified proteins and wnt pathway-activating or inhibiting fragments thereof.

Isl1 Antibodies: In some embodiments, an Isl1 modulating agent can be an antibody, which increases the activity or expression of Isl1. The term “antibody,” as used herein, refers to full-length, two-chain immunoglobulin molecules and antigen-binding portions and fragments thereof, including synthetic variants. A typical full-length antibody includes two heavy (H) chain variable regions (abbreviated herein as VH), and two light (L) chain variable regions (abbreviated herein as VL). The term “antigen-binding fragment” of an antibody, as used herein, refers to one or more fragments of a full-length antibody that retain the ability to specifically bind to a target. Examples of antigen-binding fragments include, but are not limited to: (i) a Fab fragment, a monovalent fragment consisting of the VL, VH, CL and CH1 domains; (ii) a F(ab′).sub.2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426, 1988; and Huston et al. Proc. Natl. Acad. Sci. USA 85:5879-5883, 1988). Such single chain antibodies are also encompassed within the term “antigen-binding fragment.”

Production of antibodies and antibody fragments is well documented in the field. See, e.g., Harlow and Lane, 1988. Antibodies, A Laboratory Manual. Cold Spring Harbor, N.Y.: Cold Spring Harbor Laboratory. For example, Jones et al., Nature 321: 522-525, 1986, which discloses replacing the CDRs of a human antibody with those from a mouse antibody. Marx, Science 229:455-456, 1985, discusses chimeric antibodies having mouse variable regions and human constant regions. Rodwell, Nature 342:99-100, 1989, discusses lower molecular weight recognition elements derived from antibody CDR information. Clackson, Br. J. Rheumatol. 3052: 36-39, 1991, discusses genetically engineered monoclonal antibodies, including Fv fragment derivatives, single chain antibodies, fusion proteins chimeric antibodies and humanized rodent antibodies. Reichman et al., Nature 332:323-327, 1988 discloses a human antibody on which rat hypervariable regions have been grafted. Verhoeyen, et al., Science 239:1534-1536, 1988, teaches grafting of a mouse antigen binding site onto a human antibody.

Small Molecule Drugs: In some embodiments, the Isl1 modulating agent is a small molecule drug that increases the activity and/or expression of Isl1. For example, US2008/0108090 describes identification of a GSK-3β inhibitor (BIO) that could increase the expression and activity of Isl1 in cardiac progenitor cells.

In some embodiments, such small molecule drugs can be identified using a drug screening method e.g. screening assays such as, contacting a cell with a library of small molecules and assaying for any changes in expression, function or activity of an Isl1 molecule. For example, a candidate agent may increase the expression of an auditory protein e.g. Isl1 from an essentially undetectable level to a readily detectable level. It may also increase expression to a certain degree (e.g., there may be about a 1-, 2-, or 5-fold increase in expression). See, for example, US Pub. No.: 20160061818, which describes screening assays for drugs or agents that modulate Isl1 expression, function or activity.

In some embodiments, the invention contemplates a method for identifying an effective nonpeptide small-molecule inhibitor that promotes increased Isl1 activity and/or expression in an auditory hair cell or a cell with, or that is capable of differentiating into a cell with, one or more characteristics of an auditory hair cell.

The agent(s) and/or condition(s) may act directly or indirectly on the transcriptional machinery of Isl1.

The candidate agents can be essentially any nucleic acid (e.g., a gene or gene fragment that encodes a polypeptide (e.g., a functional protein) such as a growth factor or other cytokine (e.g., an interleukin)), any polypeptide per se (which may be a full-length protein or a biologically active fragment or other mutant thereof), or any small molecule. The small molecules can include those contained within commercially available compound libraries (suppliers include Chembridge Corp (San Diego, Calif.) and ChemDiv (San Diego, Calif.)). The screening assays can be configured as “high throughput” assays to screen many such agents at once. For example, the agents and/or cells to be assessed can be presented in an array. More specifically, the candidate agent can be, for example, a nucleic acid that encodes, or a polypeptide that is, a polypeptide active in the cellular biochemical pathway of Isl1 or any pathway that may regulate Isl1 expression, function or activity. Some examples are: Notch, WNT, or Sonic hedgehog are a part (e.g., WNT1, WNT10B, WNT11, WNT13, WNT14, WNT15, WNT2, WNT2B, WNT5a, WNT7a, or WNT8B); a homolog of Notch, WNT, or Sonic hedgehog; or a biologically active fragment or other variant of Notch, WNT, or Sonic hedgehog.

### Effective Dose

Toxicity and therapeutic efficacy of the compounds and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. A subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

Generally, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In Remington's Pharmaceutical Sciences, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's “The Pharmacological Basis of Therapeutics”, 9th Ed., Hardman et al., eds., McGraw-Hill, New York, N.Y., 1996).

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

The following non-limiting Examples serve to illustrate selected embodiments of the invention and which do not limit the scope of the invention described in the claims. It will be appreciated that variations in proportions and alternatives in elements of the components shown will be apparent to those skilled in the art and are within the scope of embodiments of the present invention.

### Example 1: Vector-Mediated Delivery of Isl1 Molecules

To develop AAV-Isl1 as treatment, it was important to assess the long-term effect on hearing restoration. The hearing of CD1 mice injected in the inner ear with AAV-Isl1, was tested 7 months after injection, by which time un-injected inner ears had developed profound hearing loss. It was found that injected inner ears had sustained hearing restoration in most of frequencies (FIGS. 3A, 3B). From 1 month to 7 months, the injected inner ears experienced slight threshold shifts of around 10 dB, an indication of long-term hearing recovery. In control inner ears between one and seven months, hearing loss progressed from moderate to profound, a demonstration of the nature of age-related hearing loss.

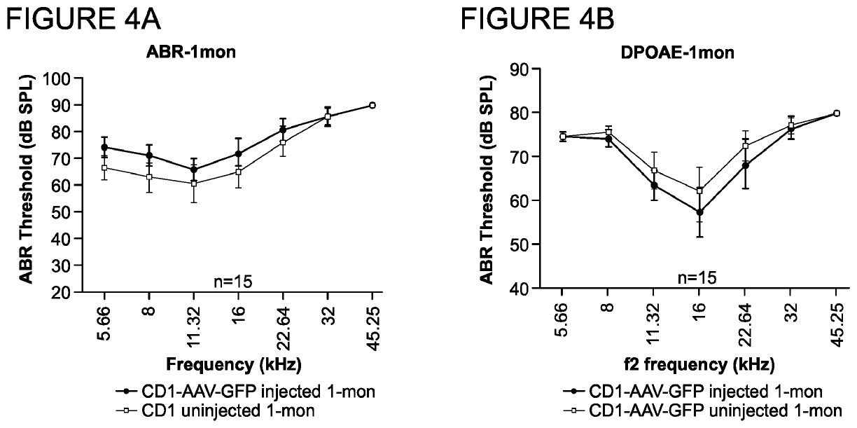

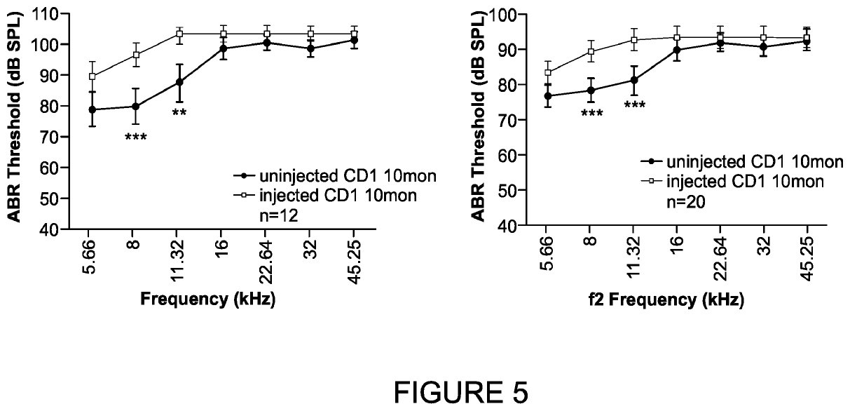

To further determine if AAV-Isl1 induced hearing recovery was Isl1 specific, neonatal inner ears were injected with AAV-GFP and a hearing study was performed one month later. Hearing recovery was not detected in the AAV-GFP injected inner ears as compared to the uninjected (FIGS. 4A, 4B). It was concluded that hearing restoration is specific to the Isl1 effect in hair cells.

In summary, the data have shown that AAV-Isl1 can restore hearing in multiple age-related hearing loss (ARHL) mouse models, and in noise-induced hearing loss (NIHL) models. The effect is Isl1 specific with the effects that can last long-term. Thus, AAV-Isl1 can serve as an ideal gene therapy to treat ARHL and NIHL.

What is claimed is:

1. A method of preventing, treating and/or reversing age-related hearing loss, noise-induced hearing loss or idiopathic hearing loss in a subject in need thereof, the method comprising:
administering to an outer and/or inner ear cell of the subject, a virus vector comprising an Islet-1 (Isl1) nucleic acid sequence wherein the Isl1 is overexpressed in the outer and/or inner ear cells as compared to expression of Isl1 in a normal outer or inner ear cell; and/or, administering cells comprising a vector encoding an Islet-1 (Isl1) nucleic acid sequence; and/or an Isl1 molecule; and/or agents comprising small molecules that activate Isl1 in inner ear cells including hair cells, thereby,
preventing, treating, and/or reversing age-related hearing loss, noise-induced hearing loss or idiopathic hearing loss in the subject.

2. The method of claim 1, wherein the Isl1 nucleic acid sequence is under control of a tissue specific promoter sequence wherein the promoter is a constitutive or inducible promoter.

3. The method of claim 1, wherein inner ear cells comprise: stria vascularis, hair cells, supporting cells or ganglion neurons.

4. The method of claim 2, wherein the tissue specific promoter sequence is a hair-cell specific promoter sequence, a stria vascularis specific promoter sequence or a supporting cell specific promoter sequence or a ganglion neuron specific promoter sequence.

5. The method of claim 1, wherein the virus vector comprises: a lentivirus, an adenovirus, an adeno-associated virus (AAV), a vesicular stomatitis virus (VSV), herpes simplex virus (HSV), vaccinia virus, pox virus, influenza virus, respiratory syncytial virus, parainfluenza virus, foamy virus or a retrovirus.

6. The method of claim 5, wherein the virus vector comprises a capsid polypeptide having a lower seroprevalence as compared to a wild-type virus.

7. The method of claim 5, wherein the virus vector is an AAV comprising a capsid polypeptide having a lower seroprevalence as compared to a wild-type AAV.

8. A method of preventing, treating, and/or reversing age-related hearing loss, noise-induced hearing loss or idiopathic hearing loss in a subject in need thereof, the method comprising:
administering to an outer and/or inner ear cell of the subject, a vector encoding an Islet-1 (Isl1) nucleic acid sequence wherein the Isl1 is overexpressed in the outer and/or inner ear cells as compared to expression of Isl1 in a normal outer or inner ear cell; and/or, administering cells comprising a vector encoding an Islet-1 (Isl1) nucleic acid sequence; and/or an Isl1 molecule; and/or Isl1 activating agents comprising small molecules, thereby,
preventing, treating, and/or reversing age-related hearing loss, noise-induced hearing loss or idiopathic hearing loss in the subject.

9. The method of claim 8, wherein inner ear cells comprise: stria vascularis, hair cells, supporting cells, or ganglion neurons.

10. The method of claim 8, the vector further comprising a tissue specific promoter sequence wherein the promoter is a constitutive or inducible promoter.

11. The method of claim 10, wherein the tissue specific promoter sequence is a hair-cell specific promoter sequence, a stria vascularis specific promoter sequence, a supporting cell specific promoter sequence or a ganglion neuron specific promoter sequence.

12. The method of claim 8, wherein the vector comprises: lentivirus vectors, adenovirus vectors, adeno-associated virus (AAV) vectors, vesicular stomatitis virus (VSV) vectors, herpes simplex virus (HSV) vectors, vaccinia virus vectors, pox virus vectors, influenza virus vectors, respiratory syncytial virus vectors, parainfluenza virus vectors, foamy virus vectors, a retrovirus vector, recombinant viral vectors, eukaryotic vectors, naked DNA vectors, plasmids, or combinations thereof.

13. The method of claim 12, wherein the vector is an AAV vector.

14. The method of claim 13, wherein the AAV vector comprises a capsid polypeptide having a lower seroprevalence in a subject as compared to a wild-type AAV vector.

15. The method of claim 8, wherein the cell comprises a stem cell, an outer ear cell, an inner ear cell, or combinations thereof.

16. A method of expressing an exogenous Islet-1 (Isl1) nucleic acid sequence in an outer and/or inner ear cell in vitro or in vivo, the method comprising: contacting the outer and/or inner ear cell with a delivery vehicle comprising an exogenous Islet-1 (Isl1) nucleic acid sequence wherein the Isl1 nucleic acid is overexpressed in the outer and/or inner ear cell as compared to expression of Isl1 in a normal cell.

17. The method of claim 16, wherein the delivery vehicle comprises: an expression vector encoding an Isl1 molecule, a recombinant viral vector encoding an Isl1 molecule, a replication-defective recombinant viral vector encoding an Isl1 molecule, a purified viral particle having a lower seroprevalence than a wild-type virus, a plasmid encoding an Isl1 molecule, a phage vector encoding an Isl1 molecule, lipids, liposomes, nanoparticles, a supercharged protein, a peptide, or any combination thereof.

18. The method of claim 17, wherein the recombinant viral vector or the replication-defective recombinant viral vector comprises: lentivirus vectors, adenovirus vectors, adeno-associated virus (AAV) vectors, vesicular stomatitis virus (VSV) vectors, herpes simplex virus (HSV) vectors, vaccinia virus vectors, pox virus vectors, influenza virus vectors, respiratory syncytial virus vectors, parainfluenza virus vectors, foamy virus vectors, or retrovirus vectors.

19. The method of claim 17, wherein the recombinant viral vector or the replication-defective recombinant viral vector is an AAV vector.

20. The method of claim 16, wherein inner ear cells comprise: stria vascularis, hair cells, supporting cells or ganglion neurons.

21. A composition comprising a virus particle comprising an Islet-1 (Isl1) nucleic acid sequence wherein the virus particle has a lower seroprevalence as compared to a wild-type virus.

22. The composition of claim 21, wherein the virus particle comprises one or more ancestral capsid polypeptides.

23. The composition of claim 21, wherein the virus particle comprises: a lentivirus, an adenovirus, an adeno-associated virus (AAV), a vesicular stomatitis virus (VSV), a herpes simplex virus (HSV), a vaccinia virus, a pox virus, an influenza virus, a respiratory syncytial virus, a parainfluenza virus, a foamy virus, or a retrovirus.

24. The composition of claim 23, wherein the virus particle is an adeno-associated virus (AAV) comprising an AAV capsid polypeptide that exhibits a lower seroprevalence than does an AAV2, AAVs/Anc80 or AAV8 capsid polypeptide or a virus particle comprising an AAV2, AAVs/Anc80 or AAV8 capsid polypeptide.

25. A method of preventing, treating and/or reversing age-related hearing loss, noise-induced hearing loss or idiopathic hearing loss in a subject in need thereof, the method comprising:
administering to an outer and/or inner ear cell of the subject, a therapeutically effective amount of an Islet-1 (Isl1) protein, peptide, mutants, or variants thereof; thereby,
preventing, treating, and/or reversing age-related hearing loss, noise-induced hearing loss or idiopathic hearing loss in the subject.

26. A method of preventing, treating and/or reversing age-related hearing loss, noise-induced hearing loss or idiopathic hearing loss in a subject in need thereof, the method comprising:
administering to an outer and/or inner ear cell of the subject, a cationic liposome comprising a therapeutically effective amount of an Islet-1 (Isl1) nucleic acid sequence; thereby,
preventing, treating and/or reversing age-related hearing loss, noise-induced hearing loss or idiopathic hearing loss in the subject.

27. A method of preventing, treating and/or reversing age-related hearing loss, noise-induced hearing loss or idiopathic hearing loss in a subject in need thereof, the method comprising:
administering to an outer and/or inner ear cell of the subject, a purified virus particle comprising an Islet-1 (Isl1) nucleic acid sequence wherein the Isl1 is overexpressed in the outer and/or inner ear cells as compared to expression of Isl1 in a normal outer or inner ear cell; thereby,
preventing, treating and/or reversing age-related hearing loss, noise-induced hearing loss or idiopathic hearing loss in the subject.

28. The method of claim 27, wherein the purified virus particle is an adeno-associated virus (AAV) comprising an AAV capsid polypeptide that exhibits a lower seroprevalence than does an AAV2, AAVs/Anc80 or AAV8 capsid polypeptide or a virus particle comprising an AAV2, AAVs/Anc80 or AAV8 capsid polypeptide.

29. The method of claim 28, wherein the purified virus particle is Anc80 according to accession number GenBank: KT235804-KT235812.

30. The method of claim 27, further comprising an Isl1 modulating agent, comprising small molecules, gene activating complexes, gene-editing complexes, oligonucleotides, siRNA, miRNA, RNAi, shRNA, peptides, antibodies, aptamers, enzymes or combinations thereof.
